(19) United States
(12) Patent Application Publication
(10) Pub. No.: US 2022/0287872 A1
(43) Pub. Date: Sep. 15, 2022

(54) DOUBLE-SHELLED CRYOLIPOLYSIS APPLICATOR

(71) Applicant: DELEO, Saint Raphael (FR)

(72) Inventor: Frédéric SAMSON, Saint Raphaël (FR)

(73) Assignee: DELEO, Saint Raphaël (FR)

(21) Appl. No.: 17/636,248

(22) PCT Filed: Aug. 18, 2020
(86) PCT No.: PCT/FR2020/051482

§ 371 Date: Feb. 17, 2022

(30) Foreign Application Priority Data
Aug. 19, 2019 (FR) 1909274

(51) Int. Cl. A61F 7/00

(52) U.S. Cl. CPC A61F 7/00; A61F 2007/0056; A61F 2007/029

(57) ABSTRACT

A cryolipolysis applicator including a receptacle made of a wall and including an opening bordered by the wall, the receptacle defining a cavity, the applicator including a pipe leading into the cavity and able to be connected to a suction system which is capable of sucking a fat fold into the cavity. The wall is made of an inner shell and of an outer shell surrounding the inner shell, the space between the inner shell and the outer shell being sealed shut with the exception of an inlet port and an outlet port, the inlet port and outlet port being connected by a circuit which extends within the space, the circuit being able to receive a cooling fluid.

This invention relates to a cryolipolysis applicator comprising a receptacle consisting of a wall and having an opening bordered by this wall, the receptacle defining a cavity, the applicator comprising a pipe leading into this cavity and able to be connected to a suction system which is capable of sucking a fat fold into the cavity.

Cryolipolysis consists of applying cold (temperature below 0° C., typically around −10° C.) to a part of the human body in order to use cold to destroy unwanted fat cells. Cryolipolysis is therefore an aesthetic and non-invasive treatment of the human body.

Cryolipolysis treatments require forming a fat fold intended to be sucked into the cavity of an applicator shaped for this application in particular. The applicator has the shape of an ovoid dome with a main axis passing through its apex, the opening of the cavity being situated in a plane perpendicular to this main axis and opposite to this apex. The fat fold is sucked into the cavity by a suction system connected to this cavity. The fat fold thus comes into contact with the surface bordering the cavity (side wall of the applicator) where it is cooled. The depth of the applicator, the texture of the skin, the thickness of the skin, . . . are all factors to take into account in order to have a cryolipolysis treatment that is effective and painless. The effectiveness of the treatment is determined by good contact between the skin of the fat fold and the cooling cavity, so that the cooling cavity can properly cool the fat fold.

The cooling of the cavity is for example carried out by a fluid circulating in a network of tunnels pierced in a block of aluminum surrounding the applicator. The tunnels are then necessarily straight since they are created by drilling, while the applicator has the shape of an ovoid dome. The tunnels are therefore only close to the applicator in some locations (tunnels tangent to the applicator). As a result, the cooling of the cavity is not optimal. In addition, the circulation of fluid in the tunnels is disrupted by the right angles where the bores meet.

Applicators in which the cooler is a Peltier system are also known. The Peltier system works by passing an electrical circuit through a linear circuit composed of a succession of fragments of two dissimilar materials. The circuit thus comprises a series of connections which are alternately colder and hotter depending on whether one is transitioning from the first material to the second material or vice versa. The circuit is shaped so that all the “cold” connections are arranged along a first plate, and all the “hot” junctions are arranged along a second plate parallel to the first plate. A “sandwich” is thus obtained with a cold face composed of the first plate and a hot face composed of the second plate, the first plate then being in contact with the wall of the applicator. This system is effective in cooling the applicator. However, such an applicator has several disadvantages: on the one hand, it is bulky because several Peltier systems are necessary to cover the majority of the surface of the applicator and each system comprises a water circuit to cool the hot plate, and on the other hand, this applicator consumes energy, each Peltier system requiring a substantial supply of electricity to operate.

There is therefore a need to improve the cooling of cryolipolysis applicators.

### DESCRIPTION OF THE INVENTION

The invention aims to provide a cryolipolysis applicator for which the cooling is carried out in the most efficient and practical manner possible.

This object is achieved by means of the fact that the wall consists of an inner shell and of an outer shell surrounding the inner shell, the space between the inner shell and outer shell being sealed shut with the exception of an inlet port and an outlet port, the inlet port and outlet port being connected by a circuit which extends within the space, the circuit being capable of receiving a cooling fluid.

By means of these arrangements, the receptacle is cooled more efficiently, regardless of its geometry, in particular if it has an ovoid shape. In addition, the receptacle has minimal bulk. Furthermore, the manufacturing cost of the applicator is reduced because its cooling does not require a device such as a Peltier system attached to the applicator. In particular, in comparison with applicators using such a Peltier system, the applicator according to the invention is lighter (therefore easier to handle) and has lower energy consumption.

Advantageously, the inner shell is made of a first material, and the outer shell is made of a second material, the first material being more thermally conductive than the second material.

The cold generated is thus preferentially directed towards the cavity of the applicator. As a result, the cooling of the fat fold situated within the cavity is optimized.

For example, the first material is a metal, and the second material is a plastic.

Advantageously, the circuit is at least partly situated within the thickness of the inner shell.

The cooling fluid is thus directly in contact with the inner shell. As a result, the transfer of cold to the cavity is optimized.

Advantageously, the circuit is at least partly situated within the thickness of the outer shell.

The cooling fluid is thus directly in contact with the inner shell.

Advantageously, the circuit extends within the majority of the space between the two shells.

Nearly all of the inner shell is thus directly cooled by the cooling fluid.

Advantageously, the sealing of the space between the two shells is achieved by a gasket seal which extends along the opening of the receptacle.

The sealing of the space is thus ensured, and the manufacture of the receptacle is simplified since only one gasket seal is necessary to achieve this sealing.

The invention will be well understood and its advantages better apparent upon reading the following detailed description of embodiments shown as non-limiting examples. The description refers to the accompanying drawings, in which:

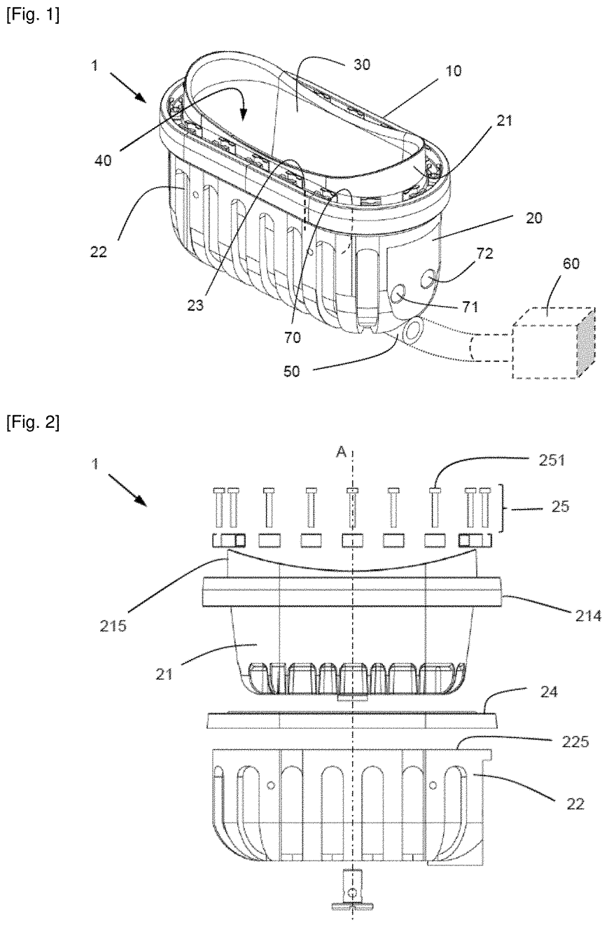

FIG. 1 is a perspective view of a cryolipolysis applicator according to the invention.

FIG. 2 is an exploded side view of the applicator of FIG. 1.

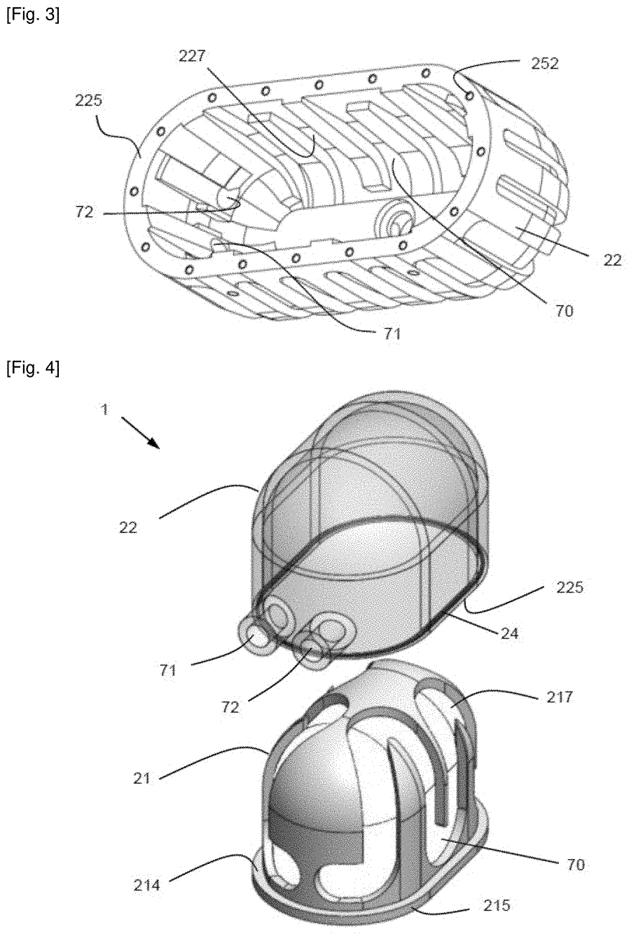

FIG. 3 is a perspective view of the outer shell of the applicator of FIG. 1.

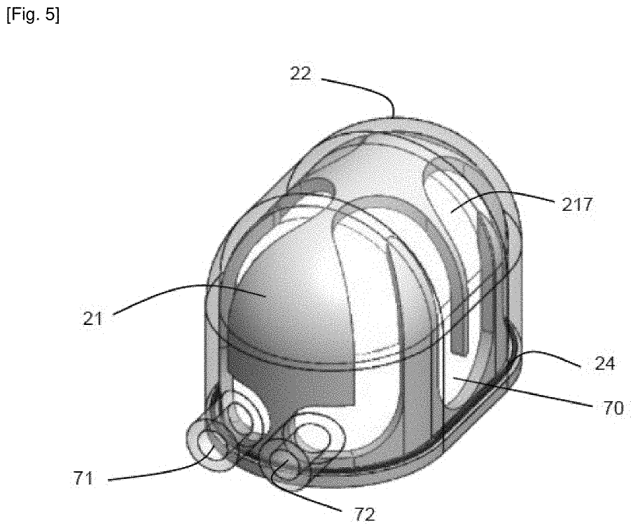

FIG. 4 is a perspective view of another embodiment of a cryolipolysis applicator according to the invention, before assembly of the inner shell and outer shell.

Fig. is a perspective view of the cryolipolysis applicator of FIG. 5, after assembly of the inner shell and outer shell (secured position).

### DETAILED DESCRIPTION OF THE INVENTION

FIG. 1 shows a cryolipolysis applicator 1. The applicator 1 comprises a receptacle 10 consisting of a wall 20. The wall 20 comprises a side wall forming a tube, and a bottom wall which extends and closes off this tube at one end. The tube is open at the other end at an opening 30 which is bordered by the wall 20. The receptacle 10 thus defines a cavity 40. The cavity 40 is intended to receive a fat fold (not shown) for treatment. For example, as represented in FIG. 1, the wall 20 has the shape of an ovoid dome, which gives the cavity 40 a shape that facilitates aspiration of a fat fold into this cavity. Advantageously, the internal surface of the wall 20 is smooth and devoid of angles, which allows the fat fold to assume the shape of this internal surface more easily.

The applicator 1 comprises a pipe 50 which leads into the cavity 40 and is able to be connected to a suction system 60 (represented by dotted lines in FIG. 1). This suction system 60 makes it possible to suck the fat fold into the cavity 40.

Referring to FIG. 1 and FIG. 2, the wall 20 is composed of an inner shell 21 and an outer shell 22 surrounding the inner shell 21. Each of these shells thus has a side wall forming an oblong tube which is open at one end at the opening 30 and curves at its opposite end into a bottom wall. Edge 215 of the inner shell 21 surrounds the opening 30 of the wall 20. Edge 225 of the outer shell 22 follows, or fits against, edge 215 of the inner shell 21. A closed space 23 is thus defined between the inner shell 21 and the outer shell 22. This space 23 is open to the outside only at an inlet port 71 and an outlet port 72. This inlet port 71 and outlet port 72 are connected by a circuit 70 which extends within the space 23. A cooling liquid serving to cool the receptacle 10 is intended to circulate in the circuit 70. The circuit 70 is described in more detail below.

The cooling liquid is chosen so as to bring and maintain the wall 20, during operation of the applicator 1, to an operating temperature below 0° C. For example, this operating temperature is between −15° C. and −3° C.

The space 23 is a sealed space, which is open only at the inlet port 71 and outlet port 72. In order to ensure this sealing, edge 215 of the inner shell 21 and edge 225 of the outer shell 22 are sealed together. For example, a gasket seal 24 extends along the periphery of the opening 30 between edge 215 of the inner shell 21 and edge 225 of the outer shell 22. This gasket seal 24 is visible in FIG. 2, which is described below.

The inner shell 21 and outer shell 22 are also secured by a securing mechanism 25.

FIG. 2 is an exploded side view of the applicator illustrated in FIG. 1. FIG. 2 thus shows the following elements, separated: the securing mechanism 25, the inner shell 21, the gasket seal 24, and the outer shell 22. These elements are represented in FIG. 2 from top to bottom along a vertical axis A (main axis A), and are assembled by translation along the main axis A. The main axis A passes through the apex of the bottom wall. Thus, the gasket seal 24 is inserted between edge 225 of the outer shell 22 and a flange 214 which runs along the entire periphery of edge 215 of the inner shell 21. The gasket seal 24 is thus sandwiched and squeezed between edge 225 of the outer shell 22 and this flange 214 when the inner shell 21 and outer shell 22 are secured by the securing mechanism 25 (secured position).

This securing mechanism 25 is composed of a set of screws 251 which are distributed along the flange of edge 215 of the inner shell 21, these screws 251 being screwed into threaded holes 252 (which are distributed along edge 225 of the outer shell 22) in order to achieve this connection. The threaded holes 252 are visible in FIG. 3. In FIG. 1, the inner shell 21 and outer shell 22 are shown in the secured position with the screws 251 screwed into the threaded holes 252. The securing mechanism 25 may be different.

The circuit 70 in which is intended to circulate the cooling liquid used to cool the wall 20 of the receptacle 10 will now be described.

The circuit 70 extends within the space 23 between the inner shell 21 and the outer shell 22, from the inlet port 71 to the outlet port 72. The fluid circulates in the circuit 70 from the inlet port 71 to the outlet port 72. Typically, these ports (71, 72) are side by side, so the circuit 70 runs all the way around the inner shell 21 about the main axis A.

Advantageously, the circuit 70 extends within the majority of the space 23, in order to follow the contours of the inner shell 21 over the largest possible portion of its surface. The inner shell 21 is thus cooled more efficiently.

For example, the circuit 70 is a pipe that winds back and forth through the space 23.

Alternatively, and advantageously, the circuit 70 is at least partly situated within the thickness of the inner shell 21, and/or at least partly within the thickness of said outer shell 22. The fluid thus circulates directly in contact with the inner shell 21, so the cooling of the inner shell 21 is more effective.

We then distinguish between three cases, the walls of the circuit 70 being formed directly by the inner shell 21 and/or the outer shell 22 in each case: in a first case, the external surface of the inner shell 21 is smooth and the outer shell 22 has on its inner face a groove 227 which forms the circuit 70, the rest (excluding the groove 227) of the outer shell 22 being in intimate contact with the surface of the inner shell 21 such that the fluid only circulates in the circuit 70. This case is illustrated in FIG. 3. The groove 227 extends from the inlet port 71 to the outlet port 72 all the way around the outer shell 22. The groove 227 runs back and forth between edge 225 and the bottom wall (or the apex) of the outer shell 22 in a series of successive “S”s so as to cover the majority of the internal surface of the outer shell 22.

In a second case, the inner face of the outer shell 22 is smooth and the inner shell 21 has on its outer face a groove 217 which forms the circuit 70, the rest (excluding the groove 217) of the inner shell 21 being in intimate contact with the surface of the outer shell 22 such that the fluid only circulates in the circuit 70, without seeping into the rest of the space between the two shells (21, 22). This case corresponds to the embodiment illustrated in FIG. 4 and FIG. 5, and which will be described below.

In a third case, the inner shell 21 has on its outer face a groove 217, and the outer shell 22 has on its inner face a groove 227 which is situated facing groove 217 when the inner shell 21 and the outer shell 22 are in the secured position. These two grooves then form a continuous conduit which is the circuit 70.

The groove (217, 227) is machined in the inner shell 21 and/or the outer shell 22. Alternatively, the inner shell 21 and/or the outer shell 22 are molded and the groove (217, 227) results from the molding.

The invention has been described above in the case where a single or double groove (217, 227) extends between the inlet port 71 and the outlet port 72. Alternatively, a plurality of grooves forming a continuous network extends between the inlet port 71 and the outlet port 72.

The inner shell 21 and the outer shell 22 are either made of the same material or are made of two different materials. In the latter case, the inner shell 21 is made of a first material, the outer shell 22 is made of a second material.

Advantageously, the first material is more thermally conductive than the second material. Indeed, the inner shell 21 then transmits cold more effectively to the fat fold situated inside the cavity 40, while the outer shell 22 contributes to maintaining the cold in the cavity 40.

For example, the first material is a metal, while the second material is a plastic (polymer) or a ceramic.

FIG. 4 and FIG. 5 illustrate an applicator 1 according to another embodiment of the invention. For clarity, the pipe 50 and the hole in the wall 20 of the receptacle 10 through which this pipe 50 leads into the cavity 40 are not shown. FIG. 4 shows the inner shell 21 and outer shell 22 in exploded perspective. FIG. 5 shows the inner shell 21 and outer shell 22 in perspective, in the secured position.

The inner face of the outer shell 22 is smooth and the inner shell 21 has on its outer face a continuous groove 217 which forms the circuit 70. The groove 217 extends from a first end to a second end. The groove 217 runs back and forth between edge 215 and the apex of the inner shell 21. The majority of the wall of the circuit 70 is therefore formed by the inner shell 21. The cooling fluid is then directly and mainly in contact with the inner shell 21, which cools the inner shell 21 more effectively. The outer shell 22 has an inlet port 71 and an outlet port 72. In the secured position, the inlet port 71 is located in line with the first end, and the outlet port 72 is located in line with the second end, such that the cooling fluid is able to flow from the inlet port 71 to the outlet port 72 through the entire groove 217.

Edge 215 of the inner shell 21 has a flange which runs along the entire periphery of this edge 215. A gasket seal 24, which extends along edge 225 of the outer shell 22, is thus sandwiched and squeezed between edge 225 of the outer shell 22 and this flange 214 when the inner shell 21 and the outer shell 22 are secured by the securing mechanism (not shown) which integrally secures the inner shell 21 to the outer shell 22.

1.-7. (canceled)

8. A cryolipolysis applicator comprising a receptacle consisting of a wall and having an opening bordered by said wall, said receptacle defining a cavity, said applicator comprising a pipe leading into said cavity and able to be connected to a suction system which is capable of sucking a fat fold into said cavity, wherein said wall consists of an inner shell and of an outer shell surrounding said inner shell, the space between said inner shell and said outer shell being sealed shut with the exception of an inlet port and an outlet port, said inlet port and said outlet port being connected by a circuit which extends within said space, said circuit being able to receive a cooling fluid.

9. The cryolipolysis applicator according to claim 8, wherein said inner shell is made of a first material, and said outer shell is made of a second material, the first material being more thermally conductive than the second material.

10. The cryolipolysis applicator according to claim 9, wherein said first material is a metal and said second material is a plastic.

11. The cryolipolysis applicator according to claim 8, wherein said circuit is at least partly situated within the thickness of said inner shell.

12. The cryolipolysis applicator according to claim 8, wherein said circuit is at least partly situated within the thickness of said outer shell.

13. The cryolipolysis applicator according to claim 8, wherein said circuit extends within the majority of said space.

14. The cryolipolysis applicator according to claim 8, wherein the sealing of the said space is achieved by a gasket seal which extends along said opening.
